(19) United States
(12) Patent Application Publication
(10) Pub. No.: US 2021/0070854 A1
(43) Pub. Date: Mar. 11, 2021

(54) ANTIMICROBIAL NANOBODIES

(71) Applicant: BOARD OF REGENTS, THE UNIVERSITY OF TEXAS SYSTEM, Austin, TX (US)

(72) Inventors: Bryan W. DAVIES, Austin, TX (US); Ashley T. TUCKER, Austin, TX (US)

(21) Appl. No.: 16/958,844

(22) PCT Filed: Dec. 28, 2018
(86) PCT No.: PCT/US18/67886

§ 371 Date: Jun. 29, 2020

Related U.S. Application Data
(60) Provisional application No. 62/611,887, filed on Dec. 29, 2017.

(51) Int. Cl. C07K 16/28

(52) U.S. Cl. CPC C07K 16/28; B82Y 5/00

(57) ABSTRACT

This application discloses a nanobody polypeptide comprising at least 2 complementarity determining regions (CDRs) and an antimicrobial peptide, wherein the polypeptide binds specifically to a microbial surface antigen. Further, this application provides a nanobody polypeptide comprising 3 CDRs and an antimicrobial peptide, wherein the polypeptide binds specifically to a microbial surface antigen.

This application claims the benefit of U.S. Provisional Patent Application No. 62/611,887, filed Dec. 29, 2017, the entirety of which is incorporated herein by reference.

This invention was made with government support under Grant No. RO1 AI125337 awarded by the National Institutes of Health. The government has certain rights in the invention.

### INCORPORATION OF SEQUENCE LISTING

The sequence listing that is contained in the file named “UTFBP1190WO_ST25.TXT”, which is 35 KB (as measured in Microsoft Windows®) and was created on Dec. 28, 2018, is filed herewith by electronic submission and is incorporated by reference herein.

### BACKGROUND OF THE INVENTION

### 1. Field of the Invention

The present invention relates generally to the field of immunology. More particularly, it concerns antibodies with antibacterial activity.

### 2. Description of Related Art

Antibiotic resistant bacteria are projected to cause 10 million deaths a year by 2050 (O'Neill, 2016). As emphasized by recent World Health Organization reports, antibiotics to treat Gram-negative bacterial infections are needed most (WHO, 2017). The path from antibiotic discovery to clinical therapy has a high attrition rate, with the last new class of antibiotics to combat Gram-negative bacteria being discovered over 40 years ago (Clatworthy et al., 2007; Payne et al., 2007). Most antibiotic screening methods have not evolved far from the innovation of Waksman's approach developed in the 1930s, and are no longer able to quickly identify new lead compounds (Lewis, 2013; Woodruff, 2014). Necessitated by the lack of new leads and sources for natural products, companies are attempting to resurrect previously unsuccessful drug candidates (Lewis, 2013). Reliable and robust antibiotic discovery platforms are urgently needed to discover new leads against new microbial targets in our arms race against resistance.

Antimicrobial polypeptides are a potent class of antimicrobials with potential to combat multi-drug resistant bacteria. Nanobodies offer a possible approached to targeted treatment of microbial infections. However, to date, reliably effective antimicrobial nanobodies have not been produced.

### SUMMARY OF THE INVENTION

In a first embodiment, there is provided a nanobody polypeptide comprising at least 2 complementarity determining regions (CDRs) and an antimicrobial peptide, wherein the polypeptide binds specifically to a microbial surface antigen. In particular aspects, the nanobody polypeptide is humanized. In a preferred aspect, the antimicrobial peptide is positioned C-terminal relative to the at least 2 CDRs.

In a further embodiment, there is provided a nanobody polypeptide comprising 3 CDRs and an antimicrobial peptide, wherein the polypeptide binds specifically to a microbial surface antigen. In particular aspects, the nanobody polypeptide is humanized. In a preferred aspect, the antimicrobial peptide is positioned C-terminal relative to the first 2 CDRs.

In some aspects, the CDRs are separated from each other and/or from the antimicrobial peptide by framework domain sequences. In particular aspects, the framework domain sequences are from immunoglobulin variable regions. In certain aspects, the framework domain sequences are from mammalian immunoglobulin variable regions. In some aspects, the framework domain sequences are from human immunoglobulin variable regions.

In certain aspects, the polypeptide binds specifically to a bacterial surface antigen. In some aspects, the bacterial surface antigen is a gram positive bacteria surface antigen. In other aspects, the bacterial surface antigen is a gram negative bacteria surface antigen.

In some aspects, the nanobody polypeptide comprises at least two CDR sequences and from N-terminus to C-terminus: a first framework domain; the first CDR; a second framework domain; the second CDR; a third framework domain; the antimicrobial peptide; and a forth framework domain. In further aspects, the nanobody comprises from N-terminus to C-terminus: a first framework domain; the first CDR; a second framework domain; the second CDR; a third framework domain; and the antimicrobial peptide.

In some aspects, the nanobody polypeptide comprises three CDR sequences and from N-terminus to C-terminus: a first framework domain; the first CDR; a second framework domain; the second CDR; a third framework domain; the antimicrobial peptide; the third CDR; and a forth framework domain. In certain aspects, the nanobody polypeptide comprises from N-terminus to C-terminus: a first framework domain; the first CDR; a second framework domain; the second CDR; a third framework domain; the third CDR; the antimicrobial peptide; and a forth framework domain. In some aspects, the nanobody polypeptide comprises from N-terminus to C-terminus: a first framework domain; the first CDR; a second framework domain; the second CDR; a third framework domain; the antimicrobial peptide; a linker sequence; the third CDR; and a forth framework domain. In certain aspects, the nanobody polypeptide comprises from N-terminus to C-terminus: a first framework domain; the first CDR; a second framework domain; the second CDR; a third framework domain; the third CDR; a linker sequence; the antimicrobial peptide; and a forth framework domain. In some aspects, the nanobody polypeptide comprises from N-terminus to C-terminus: a first framework domain; the first CDR; a second framework domain; the second CDR; a third framework domain; the third CDR; a forth framework domain; and the antimicrobial peptide.

In particular aspects, the antimicrobial peptide is positioned in place of the amino acid positions corresponding to a third CDR sequence. For example, the antimicrobial peptide can be positioned after amino acid 96 based on the Kabat antibody number convention. In some aspects, the antimicrobial peptide replaces the amino acid sequence between amino acids 96 and 108 of a nanobody, such as, SEQ ID NO: 28.

In specific aspects, the antimicrobial peptide comprises a sequence at least 80% (e.g., 81, 82, 83, 84, 85, 86, 87, 88, 89, 90, 91, 92, 93, 94, 95, 96, 97, 98, 99, or 100%) identical to the Ec5 (IHRDQQHESFLDARPEPGLTE; SEQ ID NO: 28), Ab15 (HYRALMLFHMRVRRRKL; SEQ ID NO: 29), or Cat3 (RLVRILVSKRPVAIKPYFRL; SEQ ID NO: 30). In further aspects, the antimicrobial peptide comprises the sequence of Ec5, Ab15 or Cat3 with one, two or three amino acid substitutions, insertions or deletions. In some aspects, the antimicrobial peptide is Ab15. In particular aspects, the polypeptide comprises the amino acid sequence at least 80% (e.g., 81, 82, 83, 84, 85, 86, 87, 88, 89, 90, 91, 92, 93, 94, 95, 96, 97, 98, 99, or 100%) identical to the sequence of SEQ ID NO: 1-27.

In some aspects, the polypeptide further comprises a transmembrane domain. In a further aspect, a polypeptide of the embodiments comprises two or more nanobody sequences fused together. For example, the polypeptide can be fused to at least a second nanobody polypeptide comprising at least 2 complementarity determining regions (CDRs) and an antimicrobial peptide, wherein the polypeptide binds specifically to a microbial surface antigen. In a further aspect, the polypeptide can fused to at least a second nanobody polypeptide comprising at least 3 complementarity determining regions (CDRs), wherein the polypeptide binds specifically to a microbial surface antigen.

In another embodiment, there is provided a polynucleotide molecule encoding a polypeptide of the embodiments (e.g., a nanobody polypeptide comprising at least 2 complementarity determining regions (CDRs) and an antimicrobial peptide, wherein the polypeptide binds specifically to a microbial surface antigen).

Also provided herein is a vector comprising coding sequence for the polypeptide of the embodiments (e.g., a nanobody polypeptide comprising at least 2 complementarity determining regions (CDRs) and an antimicrobial peptide, wherein the polypeptide binds specifically to a microbial surface antigen) operably linked to a promoter sequence. In some aspects, the promoter is an inducible promoter.

Further embodiments provide host cells comprising the single-chain polypeptide of the embodiments (e.g., a nanobody polypeptide comprising at least 2 complementarity determining regions (CDRs) and an antimicrobial peptide, wherein the polypeptide binds specifically to a microbial surface antigen) or a vector of the embodiments.

Another embodiment provides a pharmaceutical composition comprising an effective amount of a polypeptide of the embodiments (e.g., a nanobody polypeptide comprising at least 2 complementarity determining regions (CDRs) and an antimicrobial peptide, wherein the polypeptide binds specifically to a microbial surface antigen) in a pharmaceutically acceptable carrier.

A further embodiment provided a method of treating a microbial infection in a subject comprising administering an effective amount of a polypeptide of the embodiments (e.g., a nanobody polypeptide comprising at least 2 complementarity determining regions (CDRs) and an antimicrobial peptide, wherein the polypeptide binds specifically to a microbial surface antigen) to the subject.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B: GFP flow cytometry analysis with surface display expressing HA peptide (A) and GFP nanobody (B). A GFP nanobody (or 2×HA peptide) was expressed on the surface of E. coli. Exogenous GFP was allowed to bind to each and measured by flow cytometry. Results show that GFP bound to the GFP nanobody that was expressed on the surface of E. coli and none bound to the control bacteria expressing 2×HA peptide

FIG. 2A-B: Sequence alignment and structure of a nanobody. (A) Exemplary nanobody sequences for GFPNB (SEQ ID NO: 14) and Ab15NB (SEQ ID NO: 2) with CDR and frame work domains annotated. (B) Depicts a structural diagram of a nanobody, the isolated CDR3 domain shown in red.

FIG. 3: Surface display killing by antimicrobial nanobodies. Expression of the indicated nanobodies were induced with increasing amounts of IPTG (as indicated). Graph shows the bacterial growth following induction. The Y axis indicates optical density (OD) at 600 nm; the X axis is time (hours). The plots are as follows from the top of the graph to the bottom at 5 hours: Ab15NB No IPTG; GFPNB No IPTG; GFPNB 0.1 mM IPTG; hAb5NB No IPTG; GFPNB 1 mM IPTG; Ab15NB 0.1 mM IPTG; Ab15NB 1 mM IPTG; hAb15NB 0.1 mM IPTG; and hAb15NB 1 mM IPTG (greatest inhibition of growth).

FIG. 4A-E: Antimicrobial activity of exemplary nanobodies. (A) Top-Histogram confirms that a functional GFP nanobody expresses on the surface. There is no nonspecific binding of GFP to the outer membrane as shown in the HA Control. Bottom—GFP nanobody expression in A. baumannii 17978. No GFP binding under was observed using an Amp resistance construct, while some binding was observed with Tet resistance. (B) Growth of bacteria after IPTG induction of GFP and Ab15 nanobodies. Death of E. coli was observed over 6 hours with the Ab15NB and almost no inhibition of bacterial growth was observed with the GFPNB. (C) shows the same experiment as (B) except with the humanized nanobody scaffold and additional peptides (Ec5 and Cat3) in the CDR3 region. Some toxicity with the GFP humanized scaffold control was observed. hAb15NB is the strongest killing nanobody in this study. (D) Purification gels of Ab15NB and GFPNB with 6His tags. (E) MBCs (minimum bactericidal concentrations) showing killing of E. coli and A. baumannii using Tris/NaCl/Glucose media.

FIG. 5: Structure prediction and overlays of antimicrobial nanobody.

### DESCRIPTION OF ILLUSTRATIVE EMBODIMENTS

### I. The Present Embodiments

The present invention relates to a polypeptide construct comprising one or more nanobodies directed to one or more target microbial antigen, and methods of use thereof. In certain aspects, the nanobodies of the instant disclosure comprise and antimicrobial peptide domain. Thus, nanobody compositions of the embodiments can be used to inhabit microbial growth or to kill target microbes.

### II. Nanobodies

A nanobody, as used herein, refers to the smallest antigen binding fragment or single variable domain (“VHH”) derived from a naturally occurring heavy chain antibody and is known to the person skilled in the art. Such nanobodies can be derived from antibodies raised in Camelidae species, for example in camel, llama, dromedary, alpaca and guanaco. Nanobodies may also be synthetically produced, such as by overexpression in bacteria. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. A nanobody of the embodiments comprises at least 2 CDR domains.

The nanobodies, according to the disclosure, generally comprise a single amino acid chain that can be considered to comprise 4 “framework sequences” or FRs and 2 or 3 “complementary determining regions” or CDRs, preferably in a sequence FR1-CDR1-FR2-CDR2-FR3-(optionally CDR3)-FR4. Non-limiting examples of nanobodies of the disclosure are described in more detail further herein. It should be clear that framework regions of nanobodies may also contribute to the binding of their antigens (Desmyter et al., 2002; Korotkov et al., 2009). It should however be noted that parts, fragments, analogs or derivatives (as further described herein) of a nanobody are not particularly limited as to their length and/or size, as long as such parts, fragments, analogs or derivatives meet the further requirements outlined herein and are also preferably suitable for the purposes described herein.

The term nanobody, in its broadest sense, is not limited to a specific biological source or to a specific method of preparation. For example, the nanobodies of the disclosure can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a mammalian species, such as from a human being, or by expression of a nucleic acid encoding such a camelized VH domain; (5) by “camelization” of a “domain antibody” or “Dab,” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing.

The small size and unique biophysical properties of nanobodies excel conventional antibody fragments for the recognition of uncommon or hidden epitopes and for binding into cavities or active sites of protein targets. Further, nanobodies can be designed as bispecific and bivalent antibodies or attached to reporter molecules (Conrath et al., 2001). Nanobodies are stable and rigid single domain proteins that can easily be manufactured and survive the gastro-intestinal system. Therefore, nanobodies can be used in many applications including drug discovery and therapy (Saerens et al., 2008) but also as a versatile and valuable tool for purification, functional study and crystallization of proteins (Conrath et al., 2009).

The nanobodies or V_{HHS} may be directed against a functional conformational state of a membrane protein, as described hereinbefore. Although naive or synthetic libraries of nanobodies (for examples of such libraries, see WO9937681, WO0043507, WO0190190, WO03025020 and WO03035694) may contain conformational binders against a membrane protein in a functional conformational state.

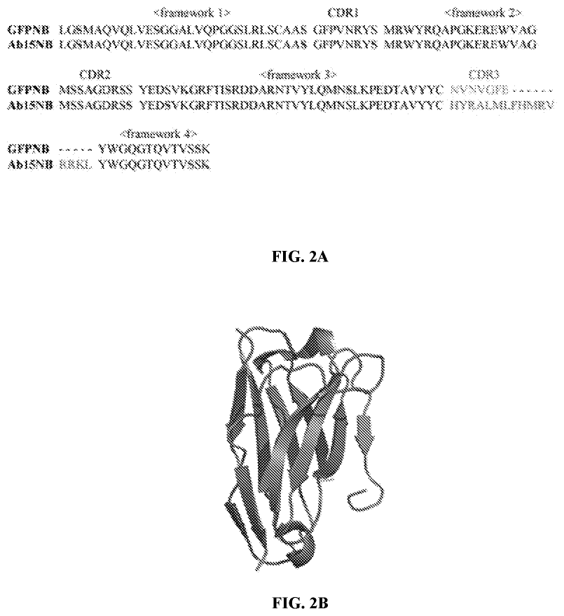

The amino acid residues of a nanobody are numbered according to the general numbering for VH domains given by Kabat et al., as applied to VHH domains from Camelids in the article of Riechmann and Muyldermans, 2000 (see for example FIG. 2 of this publication); or referred to herein. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-1.13. [In this respect, it should be noted that—as is well known in the art for VH domains and for VHH domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position. 103 according to the Kabat numbering corresponds to the start of FR4 and vice versa.

Nanobodies, and the VHH domains upon which they are based, have a number of unique structural characteristics and functional properties which make isolated VHH domains, and proteins containing the same, highly advantageous for use as functional antigen-binding domains or proteins. In particular, and without being limited thereto, VHH domains (which have been “designed” by nature to functionally bind to an antigen without the presence of, and without any interaction with, a light chain variable domain) and nanobodies can function as a single, relatively small, functional antigen-binding structural unit, domain or protein. This distinguishes the nanobodies and VHH domains from the VH and VL domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a VH domain covalently linked to a VL, domain).

Because of these unique properties, the use of nanobodies and VHH domains as single antigen-binding proteins or as antigen-binding domains (i.e. as part of a larger protein or polypeptide) offers a number of significant advantages over the use of conventional VH and VL domains, scFv's or conventional antibody fragments (such as Fab- or F(ab′)2-fragments). For example, only a single domain is required to bind an antigen with high affinity and with high selectivity, so that there is no need to have two separate domains present, nor to assure that these two domains are present in the right spatial conformation and configuration (i.e. through the use of especially designed linkers, as with scFv's). Further, nanobodies can be expressed from a single gene and require no post-translational folding or modifications. Also, nanobodies can easily be engineered into multivalent and multispecific formats. Additionally, nanobodies are highly soluble and do not have a tendency to aggregate (as with the mouse-derived “dAb's” described by Ward et al., 1989). Another highly desirable trait of nanobodies or VHH domains is that they are highly stable to heat, pH, proteases and other denaturing agents or conditions (see for example Ewert et al.). Nanobodies are easy and relatively cheap to prepare, even on a scale required for production. For example, nanobodies, VHH domains, and proteins/polypeptides containing the same can be produced using microbial fermentation (e.g. as further described below) and do not require the use of mammalian expression systems, as with for example conventional antibody fragments. Nanobodies and VHH domains are relatively small (approximately 15 kDa, or 10 times smaller than a conventional IgG) compared to conventional 4-chain antibodies and antigen-binding fragments thereof, and therefore show higher penetration into tissues (including but not limited to solid tumors and other dense tissues) than such conventional 4-chain antibodies and antigen-binding fragments thereof. Nanobodies can show so-called cavity-binding properties (inter alia due to their extended CDR3 loop, compared to conventional VH domains) and can therefore also access targets and epitopes not accessible to conventional 4-chain antibodies and antigen-binding fragments thereof. For example, it has been shown that VHH domains and Nanobodies can inhibit enzymes (see for example WO 97/49805; Transue et al., 1998; Lauwereys et al., 1998).

In additional aspects, nanobody polypeptides may be further modified by one or more other amino substitutions while maintaining their activity. In preferred aspects, such substitutions are made in the framework regions and not in the CDR domains. For example, amino acid substitutions can be made at one or more positions wherein the substitution is for an amino acid having a similar hydrophilicity. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules. Thus such conservative substitution can be made in a nanobody of the embodiments and will likely only have minor effects on their activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4). These values can be used as a guide and thus substitution of amino acids whose hydrophilicity values are within 2 are preferred, those that are within 1 are particularly preferred, and those within 0.5 are even more particularly preferred. Thus, any of the nanobody polypeptides described herein may be modified by the substitution of an amino acid, for different, but homologous amino acid with a similar hydrophilicity value. Amino acids with hydrophilicities within +/−1.0, or +/−0.5 points are considered homologous. Furthermore, it is envisioned that nanobody sequences may be modified by amino acid deletions, substitutions, additions or insertions while retaining its binding and/or antimicrobial activity.

### III. Methods of Use

The present disclosure also provides methods of using the nanobody polypeptide(s) of the present disclosure to prevent, inhibit or terminate the growth of at least one microbe which may include, for example, bacteria, archaea, fungi, algae, protozoa, multicellular parasites, and viruses.

In one embodiment, the compositions of the present disclosure provide antimicrobial effect to a target microbial organism and can be used to treat a disease or infection associated with the target microbial organism. An antimicrobial effect includes inhibiting the growth or killing of the target microbial organisms, or interfering with any biological functions of the target microbial organisms. In general, the compositions of the present disclosure can be used to treat a disease or infection at any place in a host, e.g., at any tissue including surfaces of any tissue or implant. In one embodiment, the compositions are used to specifically kill or inhibit bacterial target microbial organisms in body fluid (e.g., blood, sputum).

In some embodiments, compositions of the present disclosure are effective against bacteria including Gram-positive and Gram-negative cocci, Gram-positive and Gram-negative straight, curved and helical/vibroid and branched rods, sheathed bacteria, sulfur-oxidizing bacteria, sulfur or sulfate-reducing bacteria, spirochetes, actinomycetes and related genera, myxobacteria, mycoplasmas, rickettsias and chlamydias, cyanobacteria, archea, fungi, parasites, viruses and algae. For example, the target microbial organisms of the present disclosure include, without limitation, Escherichia coli, Candida, Salmonella, Staphylococcus, and Pseudomonas, especially Campylobacter jejuni, Candida albicans, Candida krusei, Chlamydia trachomatis, Clostridium difficile, Cryptococcus neoformans, Haempohilus influenzae, Helicobacter pylori, Moraxella catarrhalis, Neisseria gonorrhoeae, Pseudomonas aeroginosa, Salmonella typhimurium, Shigella disenteriae, Staphylococcus aureus, and Streptococcus pneumoniae. In addition, the microbial peptide composition may be used to treat chronic skin ulcers, infected acute wounds or burn wounds, infected skin eczema, impetigo, atopic dermatitis, acne, external otitis, vaginal infections, seborrhoic dermatitis, oral infections, paradontitis, conjunctivitis or pneumonia.

In particular embodiments, the compositions of the present disclosure are effective against gram-negative bacteria. Gram-positive and Gram-negative cocci include, but are not limited to, Aerococcus, Enterococcus, Halococcus, Leuconostoc, Micrococcus, Mobiluncus, Moraxella catarrhalis, Neisseria (including N. gonorrheae and N. meningitidis), Pediococcus, Peptostreptococcus, Staphylococcus species (including S. aureus, methicillin-resistant S. aureus, coagulase-negative S. aureus, and S. saprophyticus), Streptococcus species (including S. pyogenes, S. agalactiae, S. bovis, S. pneumoniae, S. mutans, S. sanguis, S. equi, S. equinus, S. thermophilus, S. morbillorum, S. hansenii, S. pleomorphus, and S. parvulus), and Veillonella.

The Gram-positive and Gram-negative straight, curved, helical/vibrioid and branched rods include, but are not limited to, Acetobacter, Acinetobacter, Actinobacillus equuli, Aeromonas, Agrobacterium, Alcaligenes, Aquaspirillum, Arcanobacterium haemolyticum, Bacillus species (including B. cereus and B. anthracis), Bacteroides species (including B. fragilis), Bartonella, Bordetella species (including B. pertussis), Brochothrix, Brucella, Burkholderia cepacia, Calymmatobacterium granulomatis, Campylobacter species (including C. jejuni), Capnocytophaga, Caulobacter, Chromobacterium violaceum, Citrobacter, Clostridium species (including C. perfringens, C. tetani and C. difficile), Comamonas, Curtobacterium, Edwardsiella, Eikenella, Enterobacter, Erwinia, Erysipelothrix, Escherichia species (including E. coli), Flavobacterium species (including E. meninosepticum), Francisella species (including E. tularensis), Fusobacterium (including E. nucleatum), Gardnerella species (including G. vaginalis), Gluconobacter, Haemophilus species (including H. influenzae and H. ducreyi), Hafnia, Helicobacter (including H. pylori), Herpetosiphon, Klebsiella species (including K. pneumoniae), Kluyvera, Lactobacillus, Legionella species (including E. pneumophila), Leptotrichia, Listeria species (including E. monocytogenes), Microbacterium, Morganella, Nitrobacter, Nitrosomonas, Pasteurella species (including P. multocida), Pectinatus, Porphyromonas gingivalis, Proteus species (including E. mirabilis), Providencia, Pseudomonas species (including E. aeruginosa, P. mallei, P. pseudomallei and E. solanacearum), Rahnella, Renibacterium salmoninarum, Salmonella, Serratia, Shigella, Spirillum, Streptobacillus species (including S. moniliformis), Vibrio species (including V. cholerae and V. vulnificus), Wolinella, Xanthobacter, Xenorhabdus, Yersinia species (including Y. pestis and Y. enter ocoliticd), Zanthomonas and Zymomonas.

The clinical diseases or infections caused by Gram-positive and/or Gram-negative bacteria, treatable with the present disclosure include abscesses, bacteremia, contamination of peritoneal dialysis fluid, endocarditis, pneumonia, meningitis, osteomyelitis, cellulitis, pharyngitis, otitis media, sinusitis, scarlet fever, arthritis, urinary tract infection, laryngotracheitis, erysipeloid, gas gangrene, tetanus, typhoid fever, acute gastroenteritis, bronchitis, epiglottitis, plague, sepsis, chancroid, wound and burn infection, cholera, glanders, periodontitis, genital infections, empyema, granuloma inguinale, Legionnaire's disease, paratyphoid, bacillary dysentary, brucellosis, diphtheria, pertussis, botulism, toxic shock syndrome, mastitis, rheumatic fever, cystic fibrosis, eye infections, plaque, and dental caries. Other uses include swine erysipelas, peritonitis, abortion, encephalitis, anthrax, nocardiosis, pericarditis, mycetoma, peptic ulcer, melioidosis, HaverhiU fever, tularemia, Moko disease, galls (e.g, crown, cane and leaf), hairy root, bacterial rot, bacterial blight, bacterial brown spot, bacterial wilt, bacterial fin rot, dropsy, columnaris disease, pasteurellosis, furunculosis, enteric redmouth disease, vibriosis offish, and fouling of medical devices.

Another embodiments of the present disclosure relates to administering a nanobody polypeptide provided herein in combination with an antibiotic. Antibiotics suitable for co-administration with the nanobody polypeptides disclosed herein include substances, produced synthetically or naturally, which can inhibit the growth of or kill microbial organisms. Such antibiotics include, without any limitation, β-lactam antibiotics (e.g., ampicillin, aziocillin, aztreonam, carbenicillin, cefoperazone, ceftriaxone, cephaloridine, cephalothin, cloxacillin, moxalactam, penicillin, piperacillin, and ticarcillin), amoxicillin, bacitracin, chloramphenicol, clindamycin, capreomycin, colistimethate, ciprofloxacin, doxycycline, erythromycin, fusidic acid, fosfomycin, fusidate sodium, gramicidin, gentamycin, lincomycin, minocycline, macrolides, monobactams, nalidixic acid, novobiocin, ofloxcin, rifamycins, tetracyclines, vancomycin, tobramycin, and trimethoprim.

Another aspect of the present disclosure relates to a composition comprising a nanobody polypeptide and an agent which can enhance, maintain, or facilitate the function or activity of the polypeptide. In one embodiment, the chemical is a protease inhibitor. The polypeptide is exposed to a protease-present environment where the presence of the protease may reduce the antimicrobial activity of the peptide via, for example, enzymatic degradation. The combination of a protease inhibitor and the polypeptide stabilizes the polypeptide from the protease degradation and thus enhances the activity of the nanobody polypeptide. The protease-present environment includes, for example, body fluid (e.g., urine, blood, serum, salvia, sputum, and mucosal fluid). The protease includes, for example, neutrophil elastase, proteinase-3, cycteine protease, metalloprotease, serine-protease, or other proteases derived from bacteria and/or hosts. The protease inhibitor includes, for example, BMF, EDTA, PMSF, benzamidine, and/or recombinant α-1 antitrypsin (rAAT).

A. Disinfectant Compositions

The nanobody polypeptides of the present disclosure are useful in a variety of environments including industrial, clinical, the household, and personal care. The polypeptide compositions of the present disclosure for industrial, pharmaceutical, household and personal care use may comprise at least one active ingredient, of which the polypeptide of the present disclosure is an active ingredient acting alone, additively, or synergistically against the target microbe.

Accordingly, the nanobody compositions of the present disclosure may be used to form contact-killing coatings or layers on a variety of substrates including personal care products (e.g., toothbrushes, contact lens cases and dental equipment), healthcare products, household products, food preparation surfaces and packaging, and laboratory and scientific equipment. Further, other substrates include medical devices such as catheters, urological devices, blood collection and transfer devices, tracheotomy devices, intraocular lenses, wound dressings, sutures, surgical staples, membranes, shunts, gloves, tissue patches, prosthetic devices (e.g., heart valves) and wound drainage tubes. Still further, other substrates include textile products such as cacpets and fabrics, paints and joint cement. A further use is as an antimicrobial soil fumigant.

B. Pharmaceutical Compositions

The nanobody polypeptides of the present disclosure may be delivered in a pharmaceutically acceptable composition. The nanobody polypeptide(s) and any suitable carrier may be prepared for delivery in forms including solution, microemulsion, suspension or aerosol.

The nanobody polypeptides of the disclosure may be incorporated into a polymer, such as, for example, a polysaccharide, a glycol polymer, a polyester, a polyurethane, a polyacrylate, a poly acrylonitrile, a polyamide, a polyolefin, a polystyrene, a vinyl polymer, a polypropylene, silk, a biopolymer, and mixtures thereof.

In the compositions of the present disclosure, the polypeptides are typically present in an amount of about 0.000001 to about 99%. In other embodiments, the polypeptides are present in an amount of about 0.001 to about 50%. In other embodiments, the polypeptides are present in an amount of about 0.01 to about 25%.

In the nanobody compositions of the present disclosure, the carrier, or mixture of carriers, is typically present in an amount of about 1 to about 99% by weight of the composition. In other embodiments, the carrier, or mixture of carriers, is typically present in an amount of about 50 to about 99% by weight of said composition. In other embodiments, the carrier, or mixture of carriers, is typically present in an amount of 75 to about 99% by weight of said composition.

Where clinical applications are contemplated, it may be necessary to prepare pharmaceutical compositions comprising proteins, antibodies, and drugs in a form appropriate for the intended application. Generally, pharmaceutical compositions may comprise an effective amount of one or more of the polypeptides of the embodiments or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition that contains at least one polypeptide of the embodiments isolated by the method disclosed herein, or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). Except insofar as any conventional carrier is incompatible with the active ingredient, its use in the pharmaceutical compositions is contemplated.

Certain embodiments of the present disclosure may comprise different types of carriers depending on whether it is to be administered in solid, liquid, or aerosol form, and whether it needs to be sterile for the route of administration, such as injection. The compositions can be administered intravenously, intrathecally, intradermally, transdermally, intrathecally, intraarterially, intraperitoneally, intranasally, intravaginally, intrarectally, intramuscularly, subcutaneously, mucosally, orally, topically, locally, by inhalation (e.g., inhalation of a nebulized formulation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference).

The nanobody polypeptides may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an antimicrobial peptide as an active ingredient. A compound may also be administered as a bolus, electuary, or paste.

Further in accordance with certain aspects of the present disclosure, the composition suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of a composition contained therein, its use in administrable composition for use in practicing the methods is appropriate. Examples of carriers or diluents include fats, oils, water, saline solutions, lipids, liposomes, resins, binders, fillers, and the like, or combinations thereof. The composition may also comprise various antioxidants to retard oxidation of one or more component. Additionally, the prevention of the action of microorganisms can be brought about by preservatives, such as various antibacterial and antifungal agents, including but not limited to parabens (e.g., methylparabens, propylparabens), chlorobutanol, phenol, sorbic acid, thimerosal or combinations thereof.

In accordance with certain aspects of the present disclosure, the composition is combined with the carrier in any convenient and practical manner, i.e., by solution, suspension, emulsification, admixture, encapsulation, absorption, and the like. Such procedures are routine for those skilled in the art.

In a specific embodiment of the present disclosure, the composition is combined or mixed thoroughly with a semi-solid or solid carrier. The mixing can be carried out in any convenient manner, such as grinding. Stabilizing agents can be also added in the mixing process in order to protect the composition from loss of therapeutic activity, i.e., denaturation in the stomach. Examples of stabilizers for use in a composition include buffers, amino acids, such as glycine and lysine, carbohydrates or lyoprotectants, such as dextrose, mannose, galactose, fructose, lactose, sucrose, maltose, sorbitol, mannitol, etc.

In some aspects, a pharmaceutical formulation comprises one or more surfactants. Surfactants used in accordance with the disclosed methods include ionic and non-ionic surfactants. Representative non-ionic surfactants include polysorbates such as TWEEN®-20 and TWEEN-80® surfactants (ICI Americas Inc. of Bridgewater, N.J.); poloxamers (e.g., poloxamer 188); TRITON® surfactants (Sigma of St. Louis, Mo.); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or (e.g., lauroamidopropyl); myristamidopropyl-, palmidopropyl-, or isostearamidopropyl-dimethylamine; sodium methyl cocoyl-, or disodium methyl oleyl-taurate; MONAQUAT™ surfactants (Mona Industries Inc. of Paterson, N.J.); polyethyl glycol; polypropyl glycol; block copolymers of ethylene and propylene glycol such as PLURONIC® surfactants (BASF of Mt. Olive, N.J.); oligo (ethylene oxide) alkyl ethers; alkyl (thio) glucosides, alkyl maltosides; and phospholipids. For example, the surfactant can be present in a formulation in an amount from about 0.01% to about 0.5% (weight of surfactant relative to total weight of other solid components of the formulation; “w/w”), from about 0.03% to about 0.5% (w/w), from about 0.05% to about 0.5% (w/w), or from about 0.1% to about 0.5% (w/w). However, in further aspects, a pharmaceutical formulation of the embodiments is essentially free of non-ionic surfactants or essentially free of all surfactants.

With respect to the therapeutic methods of the present disclosure, it is not intended that the administration of the one or more polypeptides as disclosed herein or a mutant, variant, analog or derivative thereof be limited to a particular mode of administration, dosage, or frequency of dosing; the present disclosure contemplates all modes of administration, including intramuscular, intravenous, intraperitoneal, intravesicular, intraarticular, intralesional, subcutaneous, or any other route sufficient to provide a dose adequate to treat the inflammation-related disorder. The therapeutic may be administered to the patient in a single dose or in multiple doses. When multiple doses are administered, the doses may be separated from one another by, for example, one hour, three hours, six hours, eight hours, one day, two days, one week, two weeks, or one month. For example, the therapeutic may be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject. In some embodiments, the dosage of antigen-specific T cell infusion may comprise about 100 million to about 30 billion cells, such as 10, 15, or 20 billion cells.

While the attending physician ultimately will decide the appropriate amount and dosage regimen, therapeutically effective amounts of the one or more polypeptides as disclosed herein or a mutant, variant, analog or derivative thereof may be provided at a dose of 0.0001, 0.01, 0.01 0.1, 1, 5, 10, 25, 50, 100, 500, or 1,000 mg/kg or g/kg. A typical dosage, for example is about 0.01 to about 100 mg/kg of peptide. Effective doses may be extrapolated from dose-response curves derived from in vitro or animal model test bioassays or systems.

Dosages for a particular patient or subject can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the one or more polypeptides as disclosed herein or a mutant, variant, analog or derivative thereof and the condition of the patient, as well as the body weight or surface area of the patient to be treated.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Generation and Identification of Nanobody Presenting Cells—

The anti-GFP nanobody sequence, sequences for nanobodies encoding antimicrobial peptides (GFPNB: LGSMAQVQLVESGGALVQPGGSLRLSCAASGFPVNRYSMRWYRQAPGKEREWVAG MSSAGDRSSYEDSVKGRFTISRDDARNTVYLQMNSLKPEDTAVYYCNVNVGFEYW GQGTQVTVSSK; SEQ ID NO: 14), and the sequence coding for the HA peptide (2×HA: YPYDVPDYAAYPYDVPDYAA; SEQ ID NO: 31) were synthesized by IDT, and were cloned into an IPTG-inducible LppOmpA vector and transformed into BL21(DE3) C2987 E. coli (NEB) per manufacture's instructions. Transformed E. coli was grown at 37° C., and the presence of the nanobody plasmid and insert were validated by PCR and Sanger sequencing. To evaluate surface presentation of nanobodies, flow cytometry was performed with cells presenting the anti-GFP nanobody or the HA antigen as a control, to determine GFP binding.

Surface Display Killing of Nanobodies Encoding Antimicrobial Peptides—

E. coli cells presenting the GFP nanobody, or nanobodies presenting the antimicrobial peptide Ab15 or humanized Ab15 were grown in LB supplemented with carbenicillin overnight and diluted the following day to an OD600 0.01. For cultures in which nanobody expression was induced, IPTG was added at a concentration of either 0.1 mM or 1 mM to induce nanobody expression. Cells were cultured for 6 hours following IPTG induction, and OD600 values were measured hourly. E. coli expressing humanized antibody constructs with either GFP, or antimicrobial peptides Ec5, Ab15, or Cat3.

GFPNB:
(SEQ ID NO: 14)
LGSMAQVQLVESGGALVQPGGSLRLSCAASGFPVNRYSMRWYRQAPGKER
EWVAGMSSAGDRSSYEDSVKGRFTISRDDARNTVYLQMNSLKPEDTAVYY
CNVNVGFEYWGQGTQVTVSSK
Ec5NB:
(SEQ ID NO: 11)
LGSMAQVQLVESGGALVQPGGSLRLSCAASGFPVNRYSMRWYRQAPGKER
EWVAGMSSAGDRSSYEDSVKGRFTISRDDARNTVYLQMNSLKPEDTAVYY
CIHRDQQHESFLDARPEPGLTEYWGQGTQVTVSSK
Ab15NB:
(SEQ ID NO: 2)
LGSMAQVQLVESGGALVQPGGSLRLSCAASGFPVNRYSMRWYRQAPGKER
EWVAGMSSAGDRSSYEDSVKGRFTISRDDARNTVYLQMNSLKPEDTAVYY
CHYRALMLFHMRVRRRKLYWGQGTQVTVSSK
Cat3NB:
(SEQ ID NO: 9)
LGSMAQVQLVESGGALVQPGGSLRLSCAASGFPVNRYSMRWYRQAPGKER
EWVAGMSSAGDRSSYEDSVKGRFTISRDDARNTVYLQMNSLKPEDTAVYY
CRLVRILVSKRPVAIKPYFRLYWGQGTQVTVSSK

Each of these were also evaluated for surface display killing. As above, cells transformed with the indicated nanobodies were grown with or without IPTG at a concentration of 1 mM. Optical density was measured at 2, 3.5, and 6 hours.

Protein Purification—

The sequences for GFP nanobodies and Ab15 nanobodies were cloned into the pET28a expression vector, with a non-cleavable C-terminal 6-histidine tag. The resulting plasmids were then transformed into BL21(DE3) E. coli. The presence of the appropriate plasmid and inserts were confirmed by PCR. Cell cultures harboring the expression vectors were grown to log phase (OD600 0.5-0.8) and induced by the addition of IPTG. Cultures were incubated for 6 hours. Cultures were centrifuged at 8000 rpm for 15 minutes to pellet the cells. Cells were resuspended in 20 mM Tris (pH 7.5), 200 mM NaCl, 25 mM Imidazole and lysed by sonication. Cell lysates were then centrifuged to remove insoluble debris. Cleared lysates were run over NiNTA-agarose columns by gravity flow and eluted with a 150 mL gradient of 25 mM to 500 mM imidazole. Pure Ab15 nanobodies or GFP nanobodies were pooled and dialyzed into assay buffer containing 20 mM Tris (pH 7.5), and 50 mM NaCl. Samples were stored at 4° C.

Minimum Bactericidal Concentration—

E. coli strain W3110 or A. baumannii strains Ab17978, Ab5075, or AYE were grown in Tris/NaCl/Glucose media, or the same media supplemented with GFP or CP1 nanobodies at a concentration of between 0.02 uM and 1.6 uM. Individual cultures were then subcultured onto LB agar plates and incubated overnight before evaluating the minimal bactericidal concentration of the CP1 nanobodies.

Minimum Inhibitory Concentration Determination—

E. coli strain W3110 or A. baumannii strain Ab5075 were grown in Mueller Hinton media and diluted to OD 0.001. 50 uls of each strain was added to a 96 well plate. Wells were supplemented with 50 uls of 0-32 uM GFP nanobodies or CP1 nanobodies and evaluated for plate clearing overnight at 37° C.

Computational Methods—

Sequence alignments of known nanobodies, and nanobodies disclosed herein were generated using ClustalW. Predicted structures were generated using ABodyBuilder, an automated antibody structure prediction softwar with data-driven accuracy estimation. Protein structures are depicted using PyMol.

### Example 2—Development and Characterization of Exemplary Nanobodies

Determination of Sites for Antimicrobial Peptide Insertion in Nanobodies—

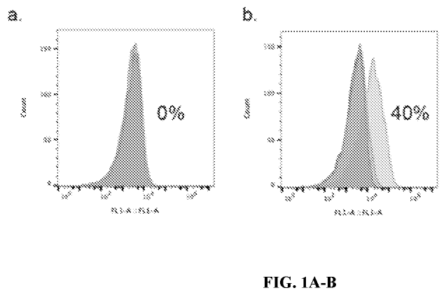

The framework and CDR sequences of a functional nanobody directed to bind GFP is shown in FIG. 2A. The CDR3 of the nanobody sequence shown in FIG. 2A was modified by the addition of the HYRALMLFHMRVRRRKL (SEQ ID NO: 29) sequence between amino acids 101 and 109 of the nanobody sequence (FIG. 2A) to generate the nanobody depicted in FIG. 1B. The predicted structure of the nanobody with the CDR3 region in red is depicted in the structure overlay of FIG. 2B.

Surface Display of Nanobodies with Antimicrobial Peptides Inhibit E. coli—

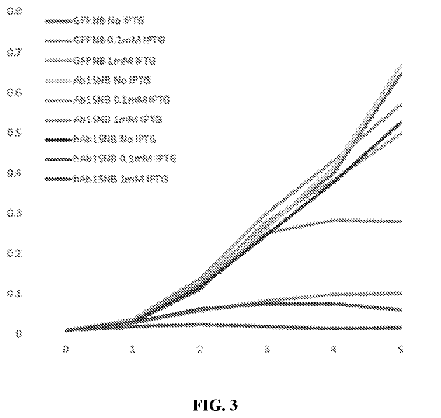

To determine whether surface display of nanobodies may have antimicrobial activity, cells which can inducibly express GFP nanobody, Ab15 nanobody, or humanized Ab15 nanobody were cultured, and then expression of the nanobodies was induced by the addition of IPTG at the indicated concentrations (FIG. 3). Induction of the GFP nanobody had little visible effect on cell growth. On the other hand, induction of Ab15 nanobody at 0.1 mM IPTG dramatically slowed cell growth after 3 hours, while 1 mM IPTG induction of Ab15 nanobody and both 0.1 mM and 1 mM IPTG induction of humanized Ab15 nanobody had a very strong inhibitory effect early in the time course (FIG. 3). Thus, both the Ab15 and hAB15 nanobodies are effective at inhibiting bacterial growth.

Cells Displaying GFP Nanobodies Bind GFP—

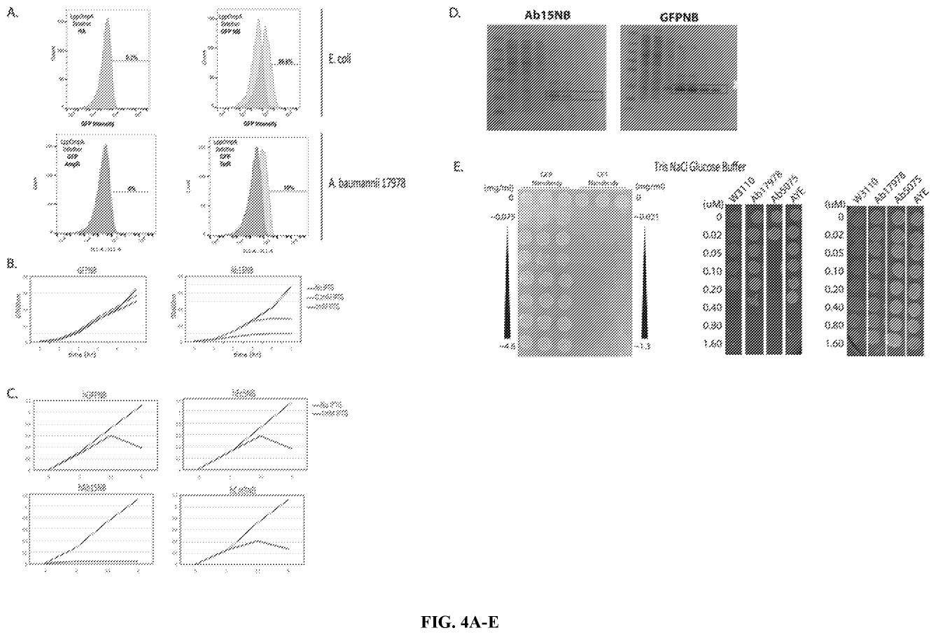

Cells transformed with GFP nanobody-LppOmpA or HA peptide-LppOmpA plasmids were subjected to GFP flow cytometry analysis (FIGS. 4A-4B). Cells expressing the HA peptide on their surface showed no binding to GFP, while almost 40% of cells expressing the GFP nanobody on the surface bound GFP. Thus, the analysis confirms that a functional GFP nanobody expresses on the surface. There is no nonspecific binding of GFP to the outer membrane as shown in the HA Control. In the A. baumannii 17978 cells, no GFP binding under was observed using an Amp resistance construct, while some binding was observed with Tet resistance.

Antimicrobial Activity of Exemplary Nanobodies—

The growth of E. coli in the presence of various nanobodies was measured. The Ab15 nanobody was confirmed to have antimicrobial activity by strong inhibition of bacterial growth when induced with either 0.1 mM or 1 mM IPTG (FIG. 4B). The hAb15 displayed even higher antimicrobial activity as observed in FIG. 4C. In addition, the hEC5 and hCat3 nanobodies displayed some inhibition of bacterial growth (FIG. 4C).

Antimicrobial Peptide Nanobodies Inhibit E. coli and A. baumannii—

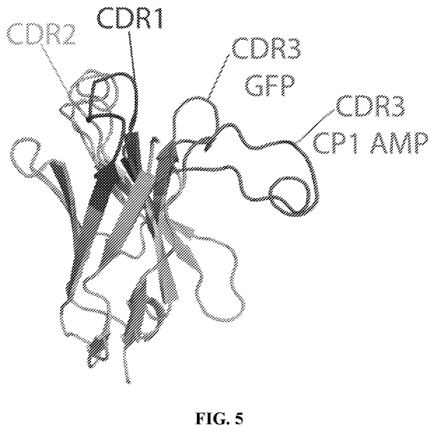

To test the efficacy of purified antimicrobial nanobodies, CP1 and GFP nanobodies were purified (FIG. 5). Cultures of wild type E. coli and several strains of A. baumannii were then treated with either CP1 nanobodies, or GFP nanobodies as a control, and plated on an antibiotic free media to assess minimal bactericidal concentration (MBC) (FIG. 4E). Treatment with GFP nanobody did not have an inhibitory effect at the concentrations tested, and saw robust growth after plating on antibiotic free agar plates. Each of the strains treated with CP1 nanobody, however, displayed significant inhibition of growth (FIG. 4E). The minimum bactericidal concentration of the CP1 nanobody was found to be about 0.40 uM for A. baumannii strain Ab17978, about 0.80 for strain AYE, and as low as about 0.05 uM for the strain Ab5075, indicating that the CP1 nanobody has significant antimicrobial activity (FIG. 4E). Thus, the nanobodies developed in the present methods are effective as antimicrobials with low minimum bactericidal concentrations.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Conrath et al., Antimicrob Agents Chemother 45: 2807, 2001.
Conrath et al., Protein Sci. 18:619, 2009.
Desmyter et al., J Biol. Chem. 277:23645, 2002.
Ewert et al., Biochemistry, 41(11):3628-3636.
International Patent Publication No. WO 97/49805
International Patent Publication No. WO0043507
International Patent Publication No. WO0190190
International Patent Publication No. WO03025020
International Patent Publication No. WO03035694
International Patent Publication No. WO9937681
Kabat et al. (“Sequence of proteins of immunological interest”, US Public Health Services, NIH Bethesda, Md., Publication No. 91.
Korotkov et al., Structure 17:255, 2009.
Kyte and Doolittle, 1982.
Lauwereys et al., EMBO J. 17(13): 3512-20, 1998.
Remington's Pharmaceutical Sciences, 18th Ed., 1990
Riechmann and Muyldermans, J. Immunol. Methods 240 (1-2): 185-195, 2000.
Saerens et al., Curr Opin Pharmacol. 8:600, 2008.
Transue et al., Proteins 32(4): 515-22, 1998.
U.S. Pat. No. 4,554,101
Ward et al., Nature 341, p. 544, 1989.

What is claimed is:

1. A nanobody polypeptide comprising at least 2 complementarity determining regions (CDRs) and an antimicrobial peptide, wherein the polypeptide binds specifically to a microbial surface antigen.

2. The polypeptide of claim 1, wherein the 2 CDRs are separated from each other by framework domain sequences.

3. The polypeptide of claim 2, wherein the framework domain sequences are from immunoglobulin variable regions.

4. The polypeptide of claim 3, wherein the framework domain sequences are from mammalian immunoglobulin variable regions.

5. The polypeptide of claim 4, wherein the framework domain sequences are from human immunoglobulin variable regions.

6. The polypeptide of claim 1, wherein the polypeptide binds specifically to a bacterial surface antigen.

7. The polypeptide of claim 1, wherein the bacterial surface antigen is a gram positive bacteria surface antigen.

8. The polypeptide of claim 1, wherein the bacterial surface antigen is a gram negative bacteria surface antigen.

9. The polypeptide of claim 1, wherein the nanobody polypeptide is humanized.

10. The polypeptide of claim 1, comprising from N-terminus to C-terminus: a first framework domain; the first CDR; a second framework domain; the second CDR; a third framework domain; the antimicrobial peptide; and a forth framework domain.

11. The polypeptide of claim 1, comprising from N-terminus to C-terminus: a first framework domain; the first CDR; a second framework domain; the second CDR; a third framework domain; and the antimicrobial peptide.

12. The polypeptide of claim 1, comprising at least 3 complementarity determining regions.

13. The polypeptide of claim 13, wherein the 3 CDRs are separated from each other by framework domain sequences.

14. The polypeptide of claim 1, comprising from N-terminus to C-terminus: a first framework domain; the first CDR; a second framework domain; the second CDR; a third framework domain; the antimicrobial peptide; the third CDR; and a forth framework domain.

15. The polypeptide of claim 1, comprising from N-terminus to C-terminus: a first framework domain; the first CDR; a second framework domain; the second CDR; a third framework domain; the third CDR; the antimicrobial peptide; and a forth framework domain.

16. The polypeptide of claim 1, comprising from N-terminus to C-terminus: a first framework domain; the first CDR; a second framework domain; the second CDR; a third framework domain; the antimicrobial peptide; a linker sequence; the third CDR; and a forth framework domain.

17. The polypeptide of claim 1, comprising from N-terminus to C-terminus: a first framework domain; the first CDR; a second framework domain; the second CDR; a third framework domain; the third CDR; a linker sequence; the antimicrobial peptide; and a forth framework domain.

18. The polypeptide of claim 1, comprising from N-terminus to C-terminus: a first framework domain; the first CDR; a second framework domain; the second CDR; a third framework domain; the third CDR; a forth framework domain; and the antimicrobial peptide.

19. The polypeptide of claim 1, wherein the antimicrobial peptide is positioned to replace the amino acids corresponding to the CDR3 of an antibody.

20. The polypeptide of claim 1, wherein the antimicrobial peptide is positioned after amino acid 96 based on the Kabat antibody number convention.

21. The polypeptide of claim 1, wherein the antimicrobial peptide replaces the amino acid corresponding to amino acids 96 and 108 of SEQ ID NO: 14.

22. The polypeptide of claim 1, wherein the antimicrobial peptide comprises a sequence at least 80% identical to the Ec5, Ab15, or Cat3.

23. The polypeptide of claim 22, wherein the antimicrobial peptide is Ab15.

24. The polypeptide of claim 1, wherein the polypeptide comprises the amino acid sequence at least 80% identical to the sequence of SEQ ID NO:1-27.

25. The polypeptide of any of claims 1-24, further comprising a transmembrane domain.

26. The polypeptide of claim 1, wherein the polypeptide is fused to at least a second nanobody polypeptide comprising at least 2 complementarity determining regions (CDRs) and an antimicrobial peptide, wherein the polypeptide binds specifically to a microbial surface antigen.

27. The polypeptide of claim 1, wherein the polypeptide is fused to at least a second nanobody polypeptide in accordance with anyone of claims 1-25.

28. The polypeptide of claim 1, wherein the polypeptide is fused to at least a second nanobody polypeptide comprising at least 3 complementarity determining regions (CDRs), wherein the polypeptide binds specifically to a microbial surface antigen.

29. A polynucleotide molecule encoding a polypeptide of any one of claims 1-28.

30. A vector comprising coding sequence for the polypeptide of any of claims 1-29 operably linked to a promoter sequence.

31. The vector of claim 30, wherein the promoter is an inducible promoter.

32. A host cell comprising the single-chain polypeptide of any of claims 1-29 or the vector of claim 30.

33. A pharmaceutical composition comprising an effective amount of a polypeptide of any one of claims 1-28 in a pharmaceutically acceptable carrier.

34. A method of treating a microbial infection in a subject comprising administering an effective amount of a polypeptide of any one of claims 1-28 to the subject.


SEQUENCE LISTING (31 sequences)

SEQ ID NO: 1 - protein, 134 aa, Artificial Sequence
  Synthetic polypeptide (Ab22NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCLLFMIQIGPNRRKRYLSLTVYWGQGTQVTVSSK

SEQ ID NO: 2 - protein, 131 aa, Artificial Sequence
  Synthetic polypeptide (Ab15NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCHYRALMLFHMRVRRRKLYWGQGTQVTVSSK

SEQ ID NO: 3 - protein, 136 aa, Artificial Sequence
  Synthetic polypeptide (AB15NB6his)
MAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCHYRALMLFHMRVRRRKLYWGQGTQVTVSSKLEHHHHHH

SEQ ID NO: 4 - protein, 141 aa, Artificial Sequence
  Synthetic polypeptide (BAPNB6his)
MAAQVQLVESGGGSVQAGGSLRLACAASASGYTESVKWMGWFRQAPGQEREGVAVISIPGGSTYYDDDVKGRFTISQDNAKNTVYLQMNSLKPEDTAMYYCAARNAGGRFRPSAAGGYNYWGQGTQVTVSSLEHHHHHHHH

SEQ ID NO: 5 - protein, 134 aa, Artificial Sequence
  Synthetic polypeptide (Br7NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCTCRTNRPCFYDLDLNVCRCSYWGQGTQVTVSSK

SEQ ID NO: 6 - protein, 134 aa, Artificial Sequence
  Synthetic polypeptide (Br9NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCYYNPLPHDCGRDNNTDICSRYWGQGTQVTVSSK

SEQ ID NO: 7 - protein, 134 aa, Artificial Sequence
  Synthetic polypeptide (Br10NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCLSVDKRPVLHPEHIYGHNHYYWGQGTQVTVSSK

SEQ ID NO: 8 - protein, 134 aa, Artificial Sequence
  Synthetic polypeptide (Cat1NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCTTSIRRRYQVSLIRRHRGKRYWGQGTQVTVSSK

SEQ ID NO: 9 - protein, 134 aa, Artificial Sequence
  Synthetic polypeptide (Cat3NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCRLVRILVSKRPVAIKPYFRLYWGQGTQVTVSSK

SEQ ID NO: 10 - protein, 145 aa, Artificial Sequence
  Synthetic polypeptide (CecrNB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCSWLSKTAKKLENSAKKRISEGIAIAIQGGPRYWGQGTQVTVSSK

SEQ ID NO: 11 - protein, 135 aa, Artificial Sequence
  Synthetic polypeptide (Ec5NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCIHRDQQHESFLDARPEPGLTEYWGQGTQVTVSSK

SEQ ID NO: 12 - protein, 125 aa, Artificial Sequence
  Synthetic polypeptide (Ec61-11NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCSNGDGTLDAGSYWGQGTQVTVSSK

SEQ ID NO: 13 - protein, 134 aa, Artificial Sequence
  Synthetic polypeptide (Ec5NB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCSNGDGTLDAGSTCAPFYARAYWGQGTQVTVSSK

SEQ ID NO: 14 - protein, 121 aa, Artificial Sequence
  Synthetic polypeptide
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSK

SEQ ID NO: 15 - protein, 126 aa, Artificial Sequence
  Synthetic polypeptide (GFPNB6His)
MAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKLEHHHHHH

SEQ ID NO: 16 - protein, 130 aa, Artificial Sequence
  Synthetic polypeptide (hAb15NB)
EVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCAHYRALMLFHMRVRRRKLYWGQGTQVTVSS

SEQ ID NO: 17 - protein, 140 aa, Artificial Sequence
  Synthetic polypeptide (hAb15NB6his)
MAEVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCAHYRALMLFHMRVRRRKLYWGQGTQVTVSSLEHHHHHH

SEQ ID NO: 18 - protein, 133 aa, Artificial Sequence
  Synthetic polypeptide (hAb15NBlpsbind)
EVQLQASGGGFVQPGGSLRLSCAASGKPTFRRLKWKMGWFRQAPGKEREFVSAISKPTFRRLKWKYYADSVKGRFTISRDNSKNTVYLQMNSLRAEDTATYYCAHYRALMLFHMRVRRRKLYWGQGTQVTVSS

SEQ ID NO: 19 - protein, 133 aa, Artificial Sequence
  Synthetic polypeptide (hCat3NB)
EVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCARLVRILVSKRPVAIKPYFRLYWGQGTQVTVSS

SEQ ID NO: 20 - protein, 143 aa, Artificial Sequence
  Synthetic polypeptide (hCat3NB6his)
MAEVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCARLVRILVSKRPVAIKPYFRLYWGQGTQVTVSSLEHHHHHH

SEQ ID NO: 21 - protein, 134 aa, Artificial Sequence
  Synthetic polypeptide (hEc5NB)
EVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCAIHRDQQHESFLDARPEPGLTEYWGQGTQVTVSS

SEQ ID NO: 22 - protein, 120 aa, Artificial Sequence
  Synthetic polypeptide (hGFPNB)
EVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCANVNVGFEYWGQGTQVTVSS

SEQ ID NO: 23 - protein, 130 aa, Artificial Sequence
  Synthetic polypeptide (hGFP6NB6his)
MAEVQLQASGGGFVQPGGSLRLSCAASGGFPVNRYSMGWFRQAPGKEREFVSAISMSSAGDRSSYYADSVKGRFTISRDNSKNTVYLQMNSLRAEDTATYYCANVNVGFEYWGQGTQVTVSSLEHHHHHH

SEQ ID NO: 24 - protein, 123 aa, Artificial Sequence
  Synthetic polypeptide (hGFPNBlpsbind)
EVQLQASGGGFVQPGGSLRLSCAASGKPTFRRLKWKMGWFRQAPGKEREFVSAISKPTFRRLKWKYYADSVKGRFTISRDNSKNTVYLQMNSLRAEDTATYYCANVNVGFEYWGQGTQVTVSS

SEQ ID NO: 25 - protein, 129 aa, Artificial Sequence
  Synthetic polypeptide (hNaLiNB6his)
MAEVQLQASGGGFVQPGGSLRLSCAASGSGSGGSGMGWFRQAPGKEREFVSAISDGSGSGSYYADSVKGRFTISRDNSKNTVYLQMNSLRAEDTATYYCASGSGGSGSGYWGQGTQVTVSSLEHHHHHH

SEQ ID NO: 26 - protein, 131 aa, Artificial Sequence
  Synthetic polypeptide (PepCNB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCILIACLGLKLLRYRRIYYWGQGTQVTVSSK

SEQ ID NO: 27 - protein, 132 aa, Artificial Sequence
  Synthetic polypeptide (ProtegrinNB)
LGSMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCRGGRLCYCRRRFCVCVGRYWGQGTQVTVSSK

SEQ ID NO: 28 - protein, 21 aa, Artificial Sequence
  Synthetic peptide
IHRDQQHESFLDARPEPGLTE

SEQ ID NO: 29 - protein, 17 aa, Artificial Sequence
  Synthetic peptide
HYRALMLFHMRVRRRKL

SEQ ID NO: 30 - protein, 20 aa, Artificial Sequence
  Synthetic peptide
RLVRILVSKRPVAIKPYFRL

SEQ ID NO: 31 - protein, 20 aa, Artificial Sequence
  Synthetic peptide
YPYDVPDYAAYPYDVPDYAA